SEQUENCE LISTING (12 sequences)

SEQ ID NO: 1 - DNA, 56 bp, Artificial Sequence
  Description of Artificial Sequence Synthetic oligonucleotide
cccggtgcagctgtttcatctcggaaacagctgcaccccccgccgccccccgtcct

SEQ ID NO: 2 - DNA, 117 bp, Artificial Sequence
  Description of Artificial Sequence Synthetic polynucleotide
gatcccgcgaaattaatacgactcactataggggaagtatttttacaacaattaccaacaacaacaacaaacaacaacaacattacattttacattctacaactacaagccaccatg

SEQ ID NO: 3 - DNA, 33 bp, Artificial Sequence
  Description of Artificial Sequence Synthetic primer
gatcccgcgaaattaatacgactcactataggg

SEQ ID NO: 4 - DNA, 36 bp, Artificial Sequence
  Description of Artificial Sequence Synthetic primer
gttcttctcctttactcatggtggcttgtagttgta

SEQ ID NO: 5 - DNA, 6119 bp, Artificial Sequence
  Description of Artificial Sequence Synthetic construct
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactataggggaattgtgagcggataacaattcccctctagaaataattttgtttaactttaagaaggagatatacatatgagtaaaggagaagaacttttcactggagttgtcccaattcttgttgaattagatggtgatgttaatgggcacaaattttctgtcagcggagagggtgaaggtgatgcaacatacggaaaacttacccttaaatttatttgcactactggaaaactacctgttccatggccaacacttgtcactactctgacgtatggtgttcaatgcttttcccgttatccggatcacatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagttaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaactgctgctgggattacacatggcatggatgagctctacaaataatgaattcgagctccgtcgacaagcttgcggccgcactcgagcaccaccaccaccaccactgagatccggctgctaacaaagcccgaaaggaagctgagttggctgctgccaccgctgagcaataactagcataaccccttggggcctctaaacgggtcttgaggggttttttgctgaaaggaggaactatatccggat

SEQ ID NO: 6 - DNA, 41 bp, Artificial Sequence
  Description of Artificial Sequence Synthetic primer
tacaactacaagccaccatgagtaaaggagaagaacttttc

SEQ ID NO: 7 - DNA, 42 bp, Artificial Sequence
  Description of Artificial Sequence Synthetic primer
gtcgacggagctcgaattcttatttgtagagctcatccatgc

SEQ ID NO: 8 - DNA, 41 bp, Artificial Sequence
  Description of Artificial Sequence Synthetic primer
tttccccgccgccccccgtcctgtcgacggagctcgaattc

SEQ ID NO: 9 - DNA, 27 bp, Artificial Sequence
  Description of Artificial Sequence Synthetic oligonucleotide
ccgctcgaccccgccgccccccgtcct

SEQ ID NO: 10 - DNA, 22 bp, Artificial Sequence
  Description of Artificial Sequence Synthetic oligonucleotide
taataagggggcggcggggaaa

SEQ ID NO: 11 - DNA, 41 bp, Artificial Sequence
  Description of Artificial Sequence Synthetic oligonucleotide
gaattcgagctccgtcgacaggacggggggcggcggggaaa

SEQ ID NO: 12 - DNA, 79 bp, Artificial Sequence
  Description of Artificial Sequence Synthetic oligonucleotide
gaattcgagctccgtcgacaagcttgcggccgcactcgagcattattattattattaaggacggggggcggcggggaaa

(19) United States
(12) Patent Application Publication
(10) Pub. No.: US 2008/0214783 A1
(43) Pub. Date: Sep. 4, 2008

(54) Method of Synthesizing Protein, mRna Immobilized on Solid Phase and Apparatus for Synthesizing Protein

(75) Inventors: Naoto Nemoto, Saitama (JP); Manish Biyani, Saitama (JP)

Correspondence Address: FOLEY AND LARDNER LLP;SUITE 500, 3000 K STREET NW, WASHINGTON, DC, 20007, US

(73) Assignees: Japan Science and Technology Agency; National Institute of Advanced Industrial Science and Technology; Janusys Co. LTD.

(21) Appl. No.: 11/718,750

(22) PCT Filed: Nov. 11, 2005
(86) PCT No.: PCT/JP2005/021175

§ 371 Date: Aug. 28, 2007

(30) Foreign Application Priority Data
Nov. 12, 2004 (JP) 2004-329493

(51) Int. Cl. C07K 1/00; C07H 21/00; B01J 19/00

(52) U.S. Cl. 530/334; 536/23.1; 422/129

(57) ABSTRACT

The present invention provides a protein synthesis method for efficiently synthesizing a desired protein so that it is properly folded so as to demonstrate a function thereof, the method comprising contacting a translation system with a solid phase-immobilized mRNA in which mRNA encoding that protein is immobilized on a solid phase, a protein synthesis apparatus for the method or the like. The protein synthesis method, protein synthesis apparatus or the like of the present invention are useful for, for example, large-volume synthesis of useful proteins.

### TECHNICAL FIELD

The present invention relates to a protein synthesis method for synthesizing a desired protein so that it is properly folded so as to demonstrate a function thereof, a solid phase-immobilized mRNA used in this synthesis method, and a protein synthesis apparatus.

### BACKGROUND ART

Continuous synthesis of cell-free translation systems according to Spirin et al. in 1988 (see A. S. Spirin, et al. (1988), Science, 242, 1162 to 1164) and subsequent cell-free translation systems resulting from modification of the wheat germ system of Endoh, et al. (see Japanese Patent Application Laid-open No. 2002-338597) have come to be practical methods for synthesizing proteins in large volume. Major pharmaceutical firms and venture corporations have recently entered this field, and research and development are being conducted targeted at various applications.

On the other hand, proteins do not function simply by being synthesized, but rather are required to be folded in the proper manner. Although research relating to this subject is also being conducted enthusiastically, in terms of technology, a typical method consists of enhancing folding efficiency by adding a protein that promotes folding referred to as a chaperone. Although various research has been conducted on the materials, conditions and so on used in cell-free translation systems (see Japanese Patent Application Laid-open Nos. H6-98790, H6-225783, H7-194, H9-291 and H7-147992), a technique for enhancing folding efficiency has yet to be developed. Consequently, under the present circumstances, in the case of synthesizing a desired protein, both proteins that are folded properly and those that are not folded properly are synthesized in large volume with conventional cell-free translation systems, and as a result, the desired protein is obtained as a protein mixture containing proteins that are folded properly. In other words, in the case of protein synthesis in accordance with conventional methods, a large amount of wasted protein ends up being synthesized.

### DISCLOSURE OF THE INVENTION

As has been described above, although cell-free translation systems have been instrumental as protein synthesis methods using various types of experimental materials, there is a need for the development of a method for efficiently synthesizing properly folded proteins so that the functions thereof are demonstrated more efficiently.

As a result of conducting extensive studies to solve the above-mentioned problems, the inventors of the present invention found that, in the case of synthesizing protein by adding a translation system after having immobilized mRNA on a solid phase, the synthesized protein is efficiently and properly folded, and the activity of the synthesized protein increases remarkably overall, thereby leading to completion of the present invention. Thus, the present invention provides a protein synthesis method as described below, a solid phase-immobilized mRNA used therein, and a protein synthesis apparatus.

(1) A protein synthesis method for synthesizing a desired protein so that the protein is properly folded so as to demonstrate a function thereof, the method comprising: contacting a translation system with a solid phase-immobilized mRNA in which the 3′-terminal of mRNA encoding that protein is immobilized on a solid phase including biotin.
(1a) A protein synthesis method for synthesizing a desired protein so that the protein is properly folded so as to demonstrate a function thereof, the method comprising: contacting a translation system with a solid phase-immobilized mRNA in which mRNA encoding that protein is immobilized on a solid phase.
(2) The protein synthesis method described in (1) above, wherein the translation system is a cell-free translation system.
(3) The protein synthesis method described in (1) of (2) above, wherein the solid phase-immobilized mRNA is immobilized on the solid phase through a linker at the 3′-terminal of the mRNA.
(4) The protein synthesis method described in any of (1) to (3) above, wherein the solid phase-immobilized mRNA is bound to the solid phase through a solid phase binding site provided on the linker.
(5) The protein synthesis method described in (3) or (4) above, wherein the linker contains, as a main backbone thereof, a polynucleotide, polyethylene, polyethylene glycol, polystyrene, peptide nucleic acid or combination thereof.
(6) The protein synthesis method described in any of (1) to (5) above, wherein the distance between the stop codon of the solid phase-immobilized mRNA and the immobilized location of the mRNA is 10 nm or less.
(7) The protein synthesis method described in any of (1) to (6) above, wherein the solid phase is selected from styrene beads, glass beads, agarose beads, Sepharose beads, magnetic beads, glass substrate, silicon substrate, plastic substrate, metal substrate, glass container, plastic container and membrane.
(8) The protein synthesis method described in any of (1) to (7), wherein the surface of the solid phase is hydrophilic.
(9) A solid phase-immobilized mRNA for synthesizing a desired protein so that the protein is properly folded so as to demonstrate a function thereof, wherein mRNA encoding the desired protein is immobilized on a solid phase through a linker.
(10) The solid phase-immobilized mRNA described in (9) above, wherein the solid phase-immobilized mRNA is immobilized on the solid phase through a linker at the 3′-terminal of the mRNA.
(11) The solid phase-immobilized mRNA described in (9) or (10) above, wherein the solid phase-immobilized mRNA is bound to the solid phase through a solid phase binding site provided on the linker.
(12) The solid phase-immobilized mRNA described in any of (9) to (11) above, wherein the linker contains, as a main backbone thereof, a polynucleotide, polyethylene, polyethylene glycol, polystyrene, peptide nucleic acid or combination thereof.
(13) The solid phase-immobilized mRNA described in any of (9) to (12) above, wherein the distance between the stop codon of the solid phase-immobilized mRNA and the immobilized location of the mRNA is 10 nm or less.
(14) The solid phase-immobilized mRNA described in any of (9) to (13) above, wherein the solid phase is selected from styrene beads, glass beads, agarose beads, Sepharose beads, magnetic beads, glass substrate, silicon substrate, plastic substrate, metal substrate, glass container, plastic container and membrane.
(15) The solid phase-immobilized mRNA described in any of (9) to (14), wherein the surface of the solid phase is hydrophilic.
(16) A protein synthesis apparatus for synthesizing a desired protein so that the protein is properly folded so as to demonstrate a function thereof, the apparatus comprising: a solid phase-immobilized mRNA in which mRNA encoding the desired protein is immobilized on a solid phase through a linker.

The protein synthesis method and so on of the present invention composed in the manner described above demonstrate, for example, the effects indicated below.

(1) A functional protein can be acquired simply by immobilizing the 3′-terminal of mRNA without having to add an expensive chaperone or other protein in particular.
(2) Although chaperones able to be used differ between prokaryotic cells and eukaryotic cells, the method of the present invention has the advantage of not limiting the type of translation system.
(3) Since the 3′-terminal of mRNA is immobilized, resistance to exonucleases can be improved.

### BRIEF DESCRIPTION OF THE DRAWINGS

FIG. 1 is a drawing showing the results of SDS-PAGE on aldehyde reductase (ALR) obtained in Example 1

FIG. 2 is a graph showing the enzyme activity of aldehyde reductase (ALR) obtained in Example 1;

FIG. 3 is a schematic drawing comparing a liquid phase protein synthesis method of the prior art with the solid phase protein synthesis of the present invention;

FIG. 4 is a schematic drawing showing the structure of a DNA construct of GFP synthesized in Example 2;

FIG. 5 is a graph showing the synthesized amounts of GFP obtained by liquid phase synthesis and solid phase synthesis carried out in Example 2;

FIG. 6 is a graph showing the activity (fluorescence intensity) of GFP obtained by liquid phase synthesis and solid phase synthesis carried out in Example 2;

FIG. 7 is a graph showing the folding efficiency of GFP obtained by liquid phase synthesis and solid phase synthesis carried out in Example 2;

FIG. 8 is a graph showing the activity (fluorescence intensity) of GFP obtained by solid phase synthesis using hydrophobic and hydrophilic solid phases carried out in Example 3;

FIG. 9 is a graph showing the activity of AKR obtained by solid phase synthesis using hydrophilic and hydrophobic solid phases and AKR obtained by liquid phase synthesis carried out in Example 3;

FIG. 10 is a drawing showing the relationship of distance d used in Example 4 between the stop codon of immobilized GFP-mRNA and the immobilized location; and

FIG. 11 is a graph showing the activity (fluorescence intensity) of GFP obtained by solid phase synthesis carried out in Example 4 using immobilized mRNA for which the distance (d) between the immobilized location of the mRNA and the stop codon varies.

### BEST MODE FOR CARRYING OUT THE INVENTION

The following provides a detailed explanation of the present invention based on embodiments thereof.

The present invention relates to a protein synthesis method for synthesizing a desired protein so that it is properly folded so as to demonstrate a function thereof. The protein synthesis method of the present invention comprises the contacting of a translation system with a solid phase-immobilized mRNA in which mRNA encoding the desired protein is immobilized on a solid phase. The present invention is based on the idea that, in the case of immobilizing one end of mRNA encoding a desired protein to a solid phase during translation of that desired protein, folding of the synthesized protein is carried out efficiently. Here, a “desired protein” refers to a specific protein targeted for synthesis. There are no particular limitations on the desired protein, and examples include proteins required for use as experimental materials such as proteins requiring analysis of a function thereof and proteins for analyzing the three-dimensional structure thereof, and useful proteins for which function has been confirmed (such as proteins used as pharmaceuticals).

Examples of target useful proteins of the present invention include cytokines such as interferon and interleukin; hormones such as insulin, glucagons, secretin, gastrin, cholecystokin, oxytocin, vasopressin, growth hormone, thyroid-stimulating hormone, prolactin, luteinizing hormone, follicle-stimulating hormone, adrenocorticotropic hormone, thyrotropin-releasing hormone, luteinizing hormone-releasing hormone, adrenocorticotropic hormone-releasing hormone, growth hormone-releasing hormone and somatostatin; opioid peptides such as endorphin, enkephalin and dynorphin; blood coagulation factors such as fibrinogen and prothrombin; enzymes such as dihydrofolic acid reductase, amyloglycosidase, amylase, invertase, isoamylase, protease, papain, pepsin, rennin, cellulase, pectinase, lipase, lactase, glucose oxidase, lysozyme, glucose isomerase, chymotrypsin, trypsin, cytochrome, seaprose, serratiopeptidase, hyaluronidase, bromelain, urokinase, hemocoagulase, thermolysin and urease; protein inhibitors such as SSI; and, proteins and various types of peptides such as albumin, globulin, globin, keratin and collagen. In this manner, the present invention offers the advantage of being able to efficiently synthesize proteins useful as pharmaceuticals and proteins useful as experimental materials in a state in which they are properly folded so as to demonstrate an inherent function thereof. Consequently, in the case of a protein useful as a pharmaceutical, the present invention offers the advantage of being able to eliminate or simplify subsequently required isolation and purification steps.

Furthermore, the “state of being properly folded so as to demonstrate a function thereof” refers to, in the case the protein is an enzyme, for example, a state of being folded so that a three-dimensional structure is adopted such that the activity of that enzyme is demonstrated.

(Solid Phase-Immobilized mRNA)

A second aspect of the present invention relates to a solid phase-immobilized mRNA (mRNA-solid phase conjugate) used to achieve solid phase protein synthesis of a desired protein such that it is properly folded so as to demonstrate a function thereof. The solid phase-immobilized mRNA of the present invention is characterized that mRNA encoding a desired protein is immobilized on a solid phase through a linker. The solid phase-immobilized mRNA used in the present invention is normally immobilized on the solid phase through a linker at the 3′-terminal of the mRNA.

The solid phase-immobilized mRNA of the present invention is normally immobilized on a solid phase through a solid phase binding site provided on this linker. Here, the “linker” is for providing a predetermined distance between the solid phase and the mRNA so as to facilitate translation, and although there are no limitations thereon provided it achieves such a function, it preferably has flexibility, hydrophilicity and a backbone having a simple structure with few side chains. More specifically, although there are no particular limitations on the linker used here, that containing as a main backbone thereof a linear substance such as a polynucleotide (including single-stranded or double-stranded DNA or RNA), a polyalkylene such as polyethylene, a polyalkylene glycol such as polyethylene glycol, a peptide nucleic acid (PNA) or polystyrene, or a combination thereof, is used preferably. Furthermore, in the present specification, “containing as a main backbone thereof” refers to containing that backbone at, for example, 60% or more, preferably 70% or more, more preferably 80% or more and most preferably 90% or more based on the total backbone length of the linker. When using a combination of the above-mentioned linear substances, they can be suitably chemically linked with a suitable linking group (such as —NH—, —CO—, —O—, —NHCO—, —CONH—, —NHNH—, —(CH₂)ₙ— (wherein, n is, for example, 1 to 10 and preferably 1 to 3), —S— or —SO—). In consideration of translation efficiency, the linker used in the present invention preferably has a length of 2 to 100 mer, more preferably 5 to 50 mer and even more preferably 10 to 30 mer. Furthermore, the linker of the present invention can be produced using a known chemical synthesis technique.

Linking of the mRNA and the linker can be carried out chemically or physically either directly or indirectly using a known technique. For example, in the case of using DNA for the linker, the linker and the mRNA can be linked by providing a sequence complementary to the terminal of the DNA linker on the 3′-terminal of the mRNA.

In order to synthesize a protein in a state in which it is properly folded so as to demonstrate an inherent function of that protein, the distance between the stop codon of the solid phase-immobilized mRNA and the immobilized location on the surface of the solid phase is preferably 20 nm or less, more preferably 15 nm or less, even more preferably 10 nm or less and particularly preferably 5 nm or less.

(Solid Phase Immobilization of mRNA)

A solid phase that serves as a carrier for immobilizing a biomolecule can be used for the solid phase used in the present invention, examples of which include beads such as styrene beads, glass beads, agarose beads, Sepharose beads or magnetic beads; substrates such as a glass substrate, silicon (quartz) substrate, plastic substrate or metal substrate (such as a gold foil substrate); containers such as a glass container or plastic container; and, membranes composed of materials such as nitrocellulose or polyvinylidene fluoride (PVDF). Beads are preferably used for the solid phase in the present invention.

The surface of the solid phase is preferably hydrophilic for synthesizing a protein in a state in which it is properly folded so as to demonstrate a function thereof. The hydrophilic solid phase surface is such that the protein is folded properly in the case of solid phase synthesis of the protein by immobilizing mRNA on the solid phase, and example of such is that having hydrophilic groups on the surface of the solid phase. Examples of hydrophilic groups include hydroxyl groups, amino groups, carboxyl groups, epoxy groups, amide groups, sodium sulfonate and sugar chains. Examples of solid phases having a hydrophilic surface include polymer beads (such as styrene beads, agarose beads or Sepharose beads) and glass beads having hydrophilic groups such as hydroxyl groups, amino groups, carboxyl groups or epoxy groups on the surface thereof.

There are no particular limitations on the method for immobilizing the solid phase-immobilized mRNA of the present invention provided that the mRNA is immobilized on the solid phase so as to not to impair translation when contacted with a translation system. Normally, a solid phase binding site is provided on a linker that links to the mRNA, and the mRNA is immobilized on the solid phase through a “solid phase binding site recognition site” where the solid phase binding site is bound to the solid phase. There are no particular limitations on the solid phase binding site provided is able to bind mRNA to a desired solid phase. For example, a molecule that specifically binds to a specific polypeptide (such as a ligand or antibody) is used as such a solid phase binding site, and in this case, the specific polypeptide that binds to that molecule is bound to the surface of a solid phase as a solid phase binding site recognition site. Examples of combinations of solid phase binding site recognition sites and solid phase binding sites include various types of receptor proteins and their ligands such as a biotin-binding protein such as avidin or streptoavidin and biotin, a maltose-binding protein and maltose, a G protein and a guanine nucleotide, a polyhistidine peptide and a metal ion such as nickel or cobalt ion, glutathione-S-transferase and glutathione, a DNA binding protein and DNA, an antibody and an antigen molecule (epitope), calmodulin and a calmodulin binding peptide, an ATP binding protein and ATP and an estradiol receptor protein and estradiol. Preferable examples of combinations of solid phase binding site recognition sites and solid phase binding sites include a biotin-binding protein such as avidin or streptoavidin and biotin, a maltose-binding protein and maltose, a polyhistidine peptide and a metal ion such as nickel or cobalt ion, glutathione-S-transferase and glutathione, and an antibody and an antigen molecule (epitope), with the combination of streptoavidin and biotin in particular being the most preferable.

Binding of the above-mentioned proteins to the surface of a solid phase can be carried out using a known method. Examples of such known methods include methods using tannic acid, formalin, glutaraldehyde, pyruvic aldehyde, bis-diazobenzidine, toluene-2,4-diisocyanate, an amino group, a carboxyl group and a hydroxyl group or an amino group (see P. M. Abdella, P. K. Smith, G. P. Royer: A New Cleavable Reagent for Cross-Linking and Reversible Immobilization of Proteins, Biochem. Biophys. Res. Commun., 87, 734 (1979)).

Furthermore, the above-mentioned combinations can be used by reversing the solid phase binding site and the solid phase binding site recognition site. Although the immobilization method described above comprises immobilization by utilizing two substances having mutual affinity, if the solid phase comprises a plastic material such as styrene beads or a styrene substrate, a portion of the linker can also be covalently bonded to the solid phase directly using a known technique (see LiquiChip Applications Handbook, Qiagen Inc.). Furthermore, in the present invention, the immobilization method is not limited to the method described above, but rather any immobilization method known among persons with ordinary skill in the art can be used.

### (Protein Solid Phase Synthesis)

According to the method of the present invention, protein is synthesized by contacting a solid phase-immobilized mRNA produced in the manner described above with a translation system (for example, by adding a translation system to the solid phase-immobilized mRNA or by adding the solid phase-immobilized mRNA to a translation system). Examples of translation systems that can be used here include both cell-free translation systems and live cell translation systems. Examples of cell-free translation systems include cell-free translation systems composed of extracts of prokaryotic or eukaryotic organisms, and for example, Escherichia coli, rabbit reticulocytes or wheat germ extract can be used (see Lamfrom, H. and Grunberg-Manago, M., Ambiguities of translation of poly U in the rabbit reticulocyte system. Biochem. Biophys Res. Commun., 1967, 27(1): 1 to 6). Examples of live cell translation systems include systems using prokaryotic or eukaryotic organisms including bacteria such as Escherichia coli.

In the present invention, a cell-free translation system is preferably used from the viewpoint of handling ease. Here, a “cell-free translation system” refers to an in vitro translation system that does not use live cells by adding materials such as amino acids required for translation to a suspension obtained by mechanically destroying the structure of the cells of a host organism. Kits that can be used for the cell-free translation system are already commercially available. For example, a cell-free translation kit containing wheat germ extract is available from Promega. In the case of using such a kit, protein synthesis can be carried out efficiently in accordance with the manual provided with the kit. In addition, various documents have been published relating to cell-free translation systems, and such documents can be referred to when carrying out the present invention (see, for example, the above-mentioned documents along with Japanese Patent Application Laid-open Nos. H6-98790, H6-225783, H7-194, H9-291, H7-147992, H7-203984, 2000-236896 and 2002-338597).

Protein synthesis using a cell-free translation system may employ a known batch method or a continuous method in which amino acids, an energy source and so on are supplied continuously (see A. S. Spirin, et al. (1988), Science, 242, 1162 to 1164). Examples of amino acids include the 20 types of L-amino acids, while examples of the energy source include adenosine 5′-triphosphate (ATP), guanosine 5′-triphosphate (GTP) and creatine phosphate. In the case of synthesizing a large amount of useful protein, a continuous method is preferable. In addition, in the case of a continuous method, a dialysis method can be used. In a dialysis method, synthesis substrates such as energy sources and amino acids are supplied to an inner dialysate through a dialysis membrane, while reaction byproducts are removed into an external dialysate.

A protein produced according to the method of the present invention can be isolated and purified from a culture (cell homogenate, culture liquid or supernatant thereof) or a solution of a cell-free translation system by using typical biochemical methods used to isolate and purify proteins, such as ammonium sulfate precipitation, gel chromatography, ion exchange chromatography or affinity chromatography, either alone or as a suitable combination thereof.

Furthermore, according to a third aspect of the present invention, the present invention provides a protein synthesis apparatus for synthesizing a desired protein so that it is properly folded so as to demonstrate a function thereof, comprising: a solid phase-immobilized mRNA in which mRNA encoding the desired protein is immobilized on a solid phase through a linker. This apparatus can be provided with, for example, an immobilization substrate immobilized with a plurality of solid-phase immobilized mRNA, a translation unit housing that immobilization substrate that carries out translation by introducing a cell-free translation system as described above, a temperature control unit for controlling the translation unit to a predetermined temperature, an energy source-amino acid source supply unit for supplying an energy source and amino acid source as described above to the translation unit, a supply path for supplying the energy source and the amino acid source to the translation unit from the energy source-amino acid source supply unit, and a protein discharge path for discharging the synthesized protein.

### EXAMPLES

The following provides a more detailed explanation of the present invention based on examples thereof. Furthermore, the present invention is not limited to these examples.

### Example 1

### Synthesis of Aldehyde Reductase Using mRNA-Immobilized Beads

### (1) Synthesis of 1n Vitro Virus (IVV) Linker—Long-Biotin-Puromycin (LBP) Linker

To begin with, the following DNA was acquired from BEX Co., Ltd.

(i) Puro-F—S[5′-(S)-TC(F)-(Spacer 18)-(Spacer 18)-(Spacer 18)-(Spacer 18)-CC-(Puro)-3′]

Here, (S) represents 5′-Thiol-Modifier C6, (Puro) represents puromycin CPG, and (Spacer 18) represents a spacer having the trade name “Spacer Phosphoramidite 18”, the chemical name (18-0-Dimethoxytritylhexaethyleneglycol, 1-[(2-cyanoethyl)-(N,N-diisopropyl)]-phosphoramidite, and the following chemical structure (all of the above are available from Glen Research Corp.).

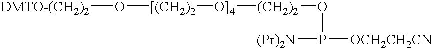

(ii) Biotin-loop [(56 mer; SEQ ID NO. 1)
5′-CCCGG TGCAG CTGTT TCATC (T-B) CGGA AACAG CTGCA
CCCCC CGCCG CCCCC CG(T)CCT-3′

Furthermore, (T) represents Amino-Modifier C6 dT, and (T-B) represents Biotin-dT (both available from Glen Research Corp.). The underlined portions indicate restrictase PvuII sites.

The LBP linker was purified after crosslinking the (i) Puro-F—S and the (ii) Biotin-loop in accordance with the following method.

10 nmol of Puro-F—S were dissolved in 100 μl of 50 mM phosphate buffer (pH 7.0) followed by the addition of 1 μl of 100 mM Tris[2-carboxyethyl] phosphine (TCEP, Pierce) (final concentration: 1 mM) and allowing to stand for 6 hours at room temperature to reduce the Thiol of the Puro-F—S. Immediately before carrying out the crosslinking reaction, the TCEP was removed using NAP5 (Amersham, 17-0853-02) equilibrated with 50 mM phosphate buffer (pH 7.0).

20 μl of 500 pmol/μl Biotin-loop and 20 μl of 100 mM crosslinking agent EMCS (344-05051; 6-maleimidohexanoic acid N-hydroxysuccinide ester), Dojindo) were added to 100 μl of 0.2 M phosphate buffer (pH 7.0) followed by stirring well, allowing to stand for 30 minutes at 37° C. and removing the unreacted EMCS. After drying the precipitate under reduced pressure, the precipitate was dissolved in 10 μl of 0.2 M phosphate buffer (pH 7.0) followed by addition of the above-mentioned reduced Puro-F—S (up to 10 nmol) and allowing to stand overnight at 4° C. After adding TCEP to the sample to a final concentration of 4 mM and allowing to stand for 15 minutes at room temperature, the unreacted Puro-F—S was removed by ethanol precipitation followed by purification by HPLC under the conditions indicated below to remove the unreacted Biotin-loop.

Column: Nacalai Tesque COSMOSIL 37918-31, 10×250 mm, C18-AR-300 (Waters)

Buffer A: 0.1 M TEAA

Buffer B: 80% acetonitrile (diluted with ultrapure water)

Flow rate: 0.5 m/min (B %:15 to 35%, 33 min)

The HPLC fractions were analyzed with 18% acrylamide gel (8 M urea, 62° C.), and after drying the target fraction under reduced pressure, it was dissolved with DEPC-treated water to a concentration of 10 pmol/μl.

### (2) Ligation Enzyme Reaction Using T4 RNA Ligase

A ligation reaction was carried out by adding 15 pmol of linker to 10 pmol of aldehyde reductase (ALR) mRNA in 201 of T4 RNA ligase buffer (50 mM Tris-HCl, pH 7.5; 10 mM MgCl₂; 10 mM DTT; 1 mM ATP). After warming at 70° C. for 5 minutes with a heating block to carry out annealing prior to addition of enzyme, the reactants were cooled at room temperature for 10 minutes and then placed on ice. 1 μl of T4 polynucleotide kinase (10 U/μl; Takara), 1.5 μl of T4 RNA ligase (40 U/μl; Takara) and 2 μl of SUPERase RNase inhibitor (20 U/μl; Ambion) were added followed by incubating for 2 hours at 25° C.

(3) Purification of mRNA-Linker

The final product of (2) above was purified using the RNeasy kit available from Qiagen Inc. in accordance with the protocol provided to remove the unlinked linker in (2) above. Moreover, the purified mRNA-linker (30 to 50 μl) was concentrated to a concentration suitable for a translation template using the Edge Biosystem nucleic acid coprecipitator in accordance with the protocol provided.

(4) Immobilization of mRNA-Linker on Streptoavidin (StAV) Beads

4 μl of streptoavidin beads (Magnotex-SA) available from Takara to 1 pmol of ALR mRNA were removed into a 1.5 ml Eppendorf tube followed by removing the supernatant after allowing to stand undisturbed for 1 minute on a magnetic stand. After washing the remaining beads with the 1× binding buffer provided at 10 times the initial volume of the beads (40 μl), the beads were additionally washed with 0.01% BSA solution. An equal volume of 2× binding buffer was mixed with ALR mRNA-linker and added to the tube containing the beads. The beads were then suspended and then incubated by rotating the tube slowly for 15 minutes at room temperature. After binding, the beads were washed twice with 10 volumes of 1× binding buffer followed by washing once with 0.01% BSA solution. This was then used for translation as immobilized mRNA.

(5) Synthesis of mRNA-Puromycin-Protein Conjugate (IVV) by Translation

After mixing, stirring and centrifuging all of the reagents, the mixture was allowed to stand undisturbed on ice. The conjugate was prepared in the manner described below in the case of 100 μl reaction scale.

2.5 μl of methionine master mix, 2.5 μl of leucine master mix, 5 μl of 1 M potassium acetate and 8 μl of SUPERase RNase inhibitor were mixed followed by slowly pipetting in 68 μl of ReticLysate to prevent the formation of bubbles (all reagents are available from Ambion).

This mixture was then transferred to a tube containing ALR mRNA-immobilized beads followed by mixing and incubating for 30 minutes at 30° C. Subsequently, 100 mM MgCl₂ and 700 mM KCl were added followed by incubating for 90 minutes at 37° C. Next, the magnetic beads were gathered on the side of the tube with a magnetic stand followed by carefully discarding the supernatant. Next, the beads were washed twice with 1× binding buffer and then washed once with 0.01% BSA solution. The beads were additionally washed with 1× PvuII buffer.

(6) Separation of IVV from StAV Beads and Separation of mRNA Portion of IVV

In order to separate the IVV synthesized on the beads synthesized in (5) above from the beads, 20 units of PvuII (Toyobo Co., Ltd.) and reaction buffer (50 mM Tris-HCl, pH 7.5) were added to the tube containing the beads to a final volume of 20 μl followed by reacting while rotating the tube slowly for 1 hour at 37° C. Next, the beads were gathered on the side of the tube with a magnetic stand and the supernatant was collected and transferred to a new tube.

The mRNA portion was separated to confirm the synthesized protein. The remaining portion consisting of the ALR protein-linker can be confirmed by SDS-PAGE since FITC (fluorescent molecule) is added to the linker. More specifically, since a DNA/RNA hybridization region is present at the linkage between the linker and the mRNA, 10 units of Tth-RNase-H (Toyobo Co., Ltd.) was added to the above-mentioned supernatant followed by incubating for 20 minutes at 40° C. This was then analyzed with 10% SDS-PAGE. The results are shown in FIG. 1. In FIG. 1, lane 1 indicates ALR synthesized using ordinary mRNA (labeled with FITC fluorescence), lane 2 indicates protein the case of synthesizing the mRNA-linker in a liquid phase, and lane 3 indicates protein in the case of synthesizing the mRNA-linker on a solid phase.

### (7) Enzyme Activity Assay

Aldehyde reductase (ALR) is an NADPH-dependent enzyme that converts NADPH to NADP when a substrate containing aldehyde is reduced. This change was measured quantitatively based on absorbance and fluorescence intensity.

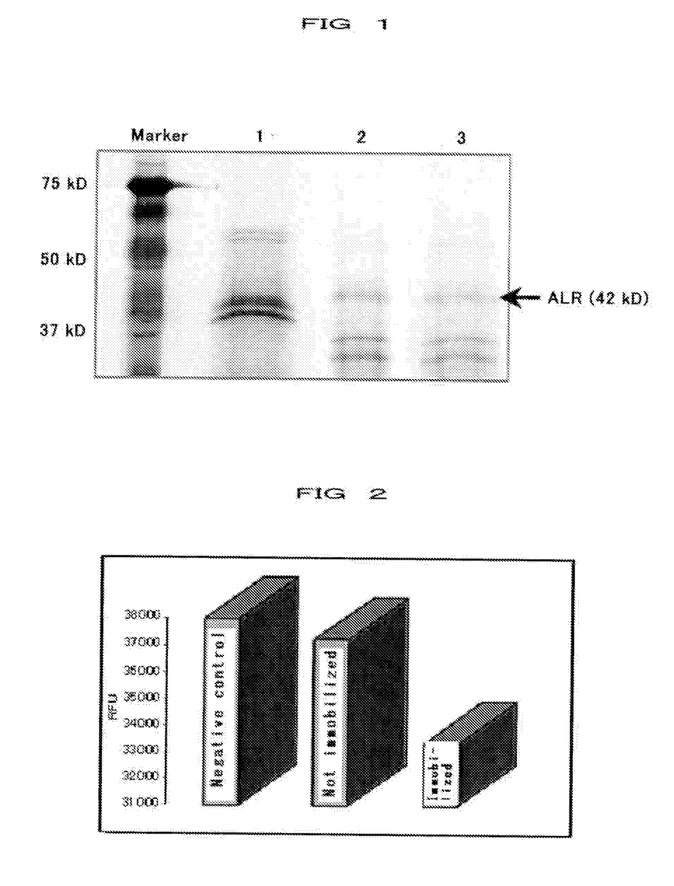

Here, the enzyme activity of ALR was analyzed fluorospectroscopically based on the enzyme-dependent decrease in NADPH having an excitation wavelength of 360 nm and a radiant wavelength of 465 nm. Fluorescence was measured at 30° C. using a fluorescence plate reader (FluPolo Microplate Reader, Takara). The supernatant of (6) above of the ALR-IVV separated from the StAV beads, and that of an equal volume as the ALR mRNA-linker immobilized on the beads were synthesized in the liquid phase without immobilizing on the beads. Subsequently, this was bound to StAV beads followed by the addition of substrate in the form of 10 mM glucuronate to the supernatant separated in accordance with (6) above and bringing to a volume of 100 μl with 50 mM potassium phosphate buffer (pH 6.5). Next, 0.2 mM NADPH was added and incubated for 10 minutes at 30° C. followed by measuring the decrease in fluorescence intensity of the NADPH using a microplate reader. More specifically, the decrease in NADPH attributable to ALR was measured on the basis of fluorescence intensity at 440 nm. Those results are shown in FIG. 2.

### (Experiment Results and Discussion)

A comparison of lane 2 (case of synthesizing the mRNA-linker in a liquid phase) and lane 3 (case of synthesizing the mRNA-linker on a solid phase) in FIG. 1 confirmed that synthesis efficiency is higher for synthesis in the liquid phase than synthesis on the solid phase. However, as shown in FIG. 2, an examination of enzyme activity revealed that enzyme activity is higher in the case of being immobilized than in the case of not being immobilized since the decrease in NADPH is larger.

### Example 2

### Synthesis of Green Fluorescent Protein (GFP) Using Immobilized mRNA

### (1) Experiment Overview

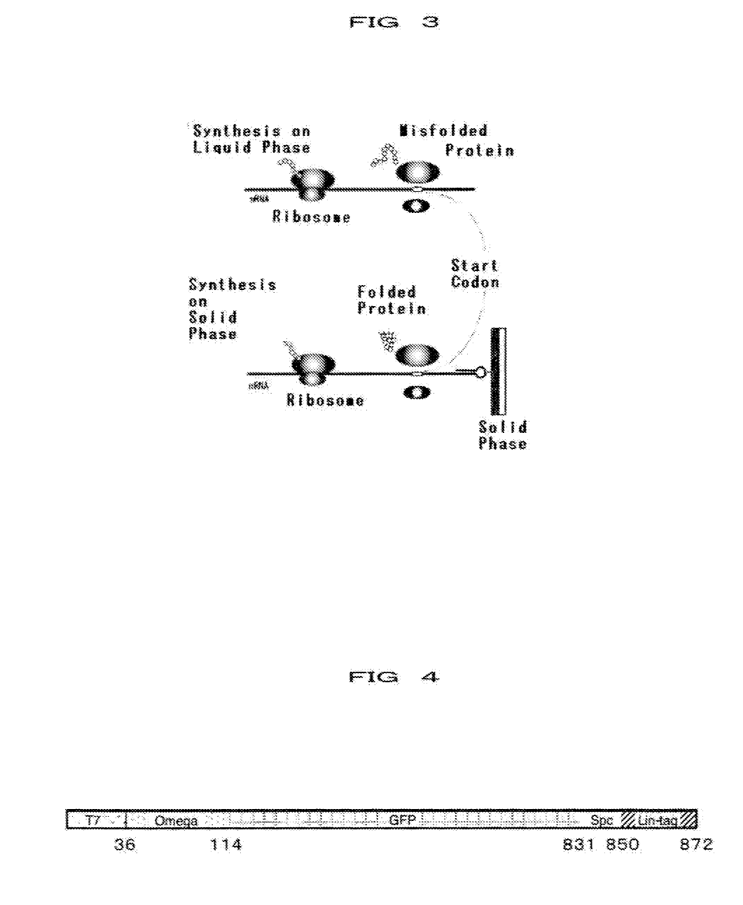

The following experiment was conducted to confirm what types of differences are present in protein folding or function between the case of synthesizing mRNA by immobilizing on a solid phase and synthesizing in a liquid phase. In the case of GFP, the degree of folding is thought to be proportional to fluorescence intensity. Consequently, mRNA encoding GFP was prepared, and, as shown in FIG. 3, the synthesized amounts of protein in the case of synthesizing by immobilizing mRNA on a solid phase and synthesizing in a liquid phase as in the prior art, and the amount of expressed function as determined by fluorescence intensity, were compared.

### (2) Preparation of DNA Construct

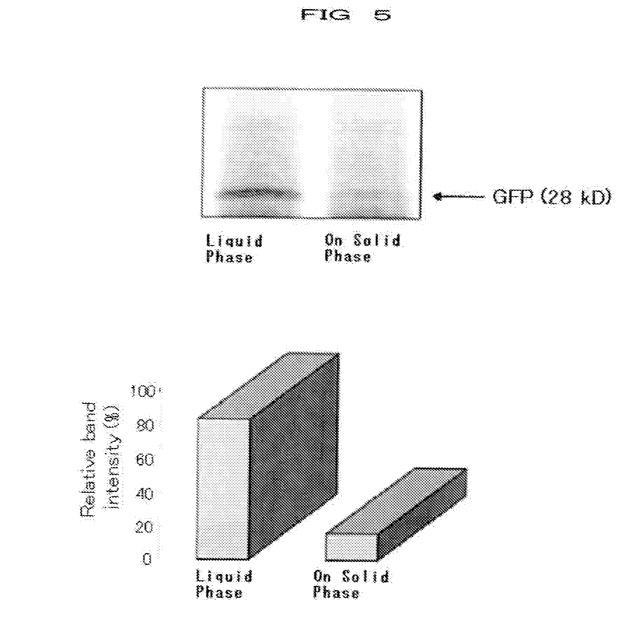

A construct for expressing GFP was prepared as shown in FIG. 4 having a T7 promoter region, a 5′ UTR (Omega) required for translation, a linker region (Spc) on the 3′ side, and a complementary sequence to biotinated DNA for immobilizing the mRNA (Lin-tag). The following template DNA (a) and primers (b) and (c) were synthesized using a DNA synthesizer to prepare this construct.

(SEQ ID NO. 2: 117 mer)

| | |
|---|---|
| (a) | GATCCCGCGAAATTAATACGACTCACTATAGGGGAAGTATTTTTACA |
| | ACAATTACCAACAACAACAACAAACAACAACAACATTACATTTTACA |
| | TTCTACAACTACAAGCCACCATG |

(SEQ ID NO. 3: 33 mer)

| | |
|---|---|
| (b) | GATCCCGCGAAATTAATACGACTCACTATAGGG |

(SEQ ID NO 4: 36 mer)

| | |
|---|---|
| (c) | GTTCTTCTCCTTTACTCATGGTGGCTTGTAGTTGTA |

Next, PCR was carried out using the above-mentioned DNA template (a) and the primers (b) and (c) under conditions consisting of (1) annealing for 30 seconds at a temperature of 69° C., (2) elongation for 40 seconds at a temperature of 72° C., and (3) denaturation for 30 seconds at a temperature of 95° C. repeated for 30 cycles.

In addition, PCR was carried out using a plasmid pET-21a(+) (SEQ ID NO. 5), which encodes a mutant GFPwt5 of GFP (used with the permission of Mr. Ichiro Itoh, College of Engineering, Osaka University), as a template along with the following primers (d) and (e).

(SEQ ID NO. 6: 41 mer)

| | |
|---|---|
| (d) | TACAACTACAAGCCACCATGAGTAAAGGAGAAGAACTTTTC |

(SEQ ID NO. 7: 42 mer)

| | |
|---|---|
| (e) | GTCGACGGAGCTCGAATTCTTATTTGTAGAGCTCATCCATGC |

PCR was carried out under conditions consisting of (1) annealing for 30 seconds at a temperature of 69° C., (2) elongation for 40 seconds at a temperature of 72° C., and (3) denaturation for 30 seconds at a temperature of 95° C. repeated for 30 cycles.

Next, the sequence of the Lin-tag was further connected to the resulting PCR product. In order to do this, PCR was carried out by using the PCR product obtained in the previous step as a template along with the above-mentioned primer (d) and the following primer (f).

(SEQ ID NO. 8: 41 mer)

| | |
|---|---|
| (f) | TTTCCCCGCCgCCCCccGTCCTGTCGACGGAGCTCGAATTC |

PCR was carried out under conditions consisting of (1) annealing for 30 seconds at a temperature of 69° C., (2) elongation for 40 seconds at a temperature of 72° C., and (3) denaturation for 30 seconds at a temperature of 95° C. repeated for 30 cycles.

After carrying out about 10 cycles of PCR at an annealing temperature of 55° C. and in the absence of primer on the PCR product obtained in this manner and the PCR product synthesized using the above-mentioned (a) to (c), annealing for 30 seconds at a temperature of 69° C., elongation for 40 seconds at a temperature of 72° C., and denaturation for 30 seconds at a temperature of 95° C. were repeated for 30 cycles using (b) and (f).

### (3) Transcription

After purifying the PCR product prepared in (2) above by phenol extraction and ethanol precipitation, RNA was synthesized using the RiboMax transcription kit (Promega) in accordance with the protocol provided.

(4) Linker DNA for Immobilizing mRNA

DNA inserted with a nucleotide containing biotin as indicated below was synthesized to bind the mRNA synthesized in (3) above to a solid phase at the 3′-terminal.

(SEQ ID NO. 9)

| | |
|---|---|
| (g) | 5′-CCGC(T-B)CGACCCCGCCGCCCCCCGTCCT-3′ |

Here, (T-B) indicates a biotinated thymine nucleotide.

2 pmol of the GFP-mRNA prepared in (2) above and 3 pmol of the DNA of (g) were added to a microtube followed by the addition of 2 μl of 10×T4 RNA ligase buffer in the form of the T4 ligase addition buffer available from Takara and 1.2 μl of 0.1% BSA followed by bringing to a volume of 20 μl with sterilized water. After mixing well, the mixture was incubated for 5 minutes at 80° C. and slowly cooled over the course of 30 minutes to 10° C. Next, 1 μl (10 units/μl) of T4 polynucleotide kinase (NEB), 1.5 μl (40 U/μl) of T4 RNA ligase and 2 μl (20 U/μl) of SUPERase RNase inhibitor were added followed by incubating for 1 hour at 25° C.

(5) Immobilization of mRNA on Streptoavidin (StAV) Beads

Prior to translation, the mRNA with linker of (4) above was bound to streptoavidin magnetic beads by biotin-avidin binding as indicated below.

(i) 201 of Magnotex-SA beads (Takara) were added to 1.5 ml.

(ii) After allowing to stand undisturbed for 1 minute on a magnetic stand, the supernatant was discarded.

(iii) After washing the beads with 1× binding buffer at 10 times the initial volume of the beads, the buffer was carefully removed using a magnetic stand.

(iv) The conjugate of (4) above along with an equal volume of 2× binding buffer were mixed and added to the tube containing the beads washed in (iii) above.

(v) The beads were then suspended and incubated for 15 minutes at room temperature using a rotor.

(vi) After discarding the supernatant with a magnetic stand, the beads were washed twice with 10 volumes of 1× binding buffer containing 0.01% BSA. These beads were then used as a translation template.

### (6) Translation Using Wheat Germ Cell-Free Translation System

A wheat germ cell-free translation system (Product ID No. L4380, Promega) was used for the cell-free translation system. Translation was carried out in accordance with the protocol provided. Furthermore, two types of translation were carried out consisting of (a) the case of translating with immobilized mRNA and (b) the case of translating in a liquid phase for comparison purposes.

(a) Case of Translating with Immobilized mRNA

25 μl of wheat germ lysate were added to 4 μl of an amino acid mixture, 5 μl of 1 M potassium acetate and 3 μl of SUPERase RNase inhibitor and mixed well followed by adding to the mRNA-immobilized beads prepared in (5) above. Sterilized water was then added to a final volume of 50 μl. Next, the reactants were allowed to react for 15 minutes at 25° C. while continuously suspending the beads with a rotor.

(b) Case of Translating in a Liquid Phase

Although the composition was the same as that of (a) above, 2 pmol of linker-less mRNA encoding GFP were added instead of the mRNA immobilized on the beads followed by reacting for 15 minutes at 25° C. in an ordinary incubator without using a rotor.

### (7) Comparison of Synthesized Amounts of GFP

In order to investigate the expressed amounts of GFP, 0.5 μl of FluoroTect (Promega) were added to the composition of (6) above as a fluorescent label of the protein synthesized in the cell-free translation system. 10 μl of 2×SDS sample buffer (4% SDS, 8 M urea) were added to 6 μl of synthesized sample. Next, the mixture was incubated for 5 minutes at 70° C. to completely denature the GFP protein. After phoresing with 10% SDS-PAGE, the phoresed protein was read with a fluorescent gel imager (Typhoon, Amersham) to measure the band intensity of the labeled GFP protein, and intensity was determined using the image analysis software provided. Those results are shown in FIG. 5.

As shown in FIG. 5, the amount of GFP synthesized on the solid phase was determined to be 0.15 (±0.05) as compared with the liquid phase based on the intensity of fluorescent intensity. In addition, in the case of the immobilized mRNA, since the beads ended up aggregating non-specifically and were unable to be effectively suspended, translation did not proceed efficiently. However, this is believed to be due to the use of a (transparent) wheat germ cell-free translation system to measure the fluorescence intensity of the GFP, and it is thought that the occurrence of such problems would be unlikely when using rabbit reticulocytes.

### (8) Comparison of Expressed Amounts of GFP Function

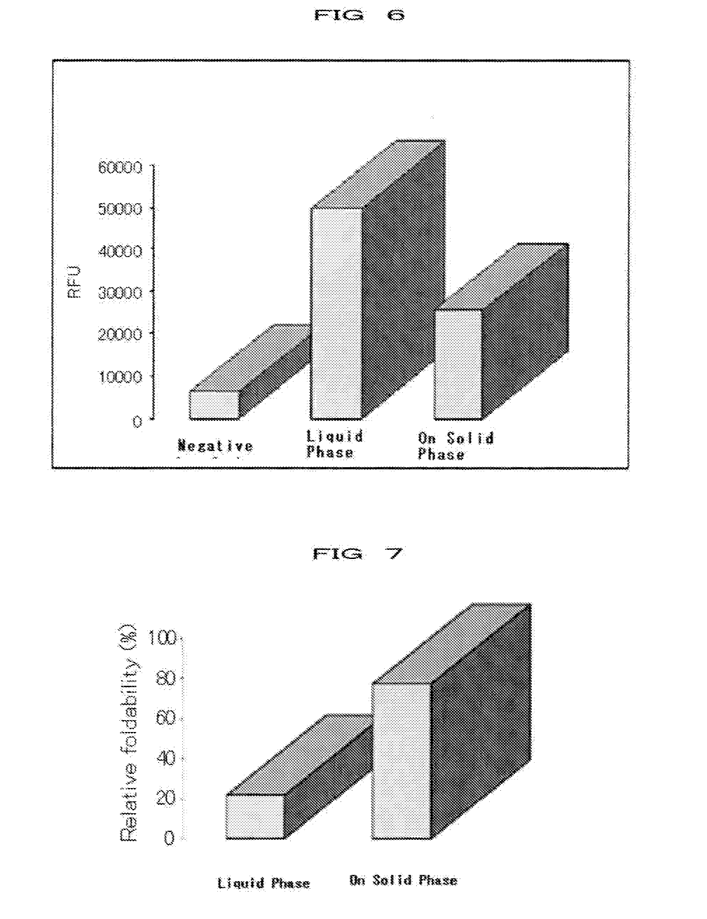

Fluorescence of the synthesis products respectively synthesized in (6) above was measured with a fluorescence microreader to confirm GFP folding. 60 μl of 10 mM Tris-HCl (pH 8.0) were added to 40 μl of each GFP translation product followed by exciting at 485 nm and measuring with a microplate reader at an emission wavelength of 535 nm. A reaction product obtained without adding mRNA was measured as a negative control. Those results are shown in FIG. 6.

As shown in FIG. 6, the relative intensity of the GFP on the solid phase was determined to be 0.52 times that of the GFP synthesized in the liquid phase.

### (9) Comparison of GFP Folding Efficiency

GFP folding efficiency was calculated based on the results of (7) and (8) above, and those results are shown in FIG. 7. According to those results, the folding efficiency of protein on the solid phase was determined to be 3.47 times higher than that of the liquid phase.

### Example 3

### Effects of Bead Surface on Translation Using Immobilized mRNA

An investigation was conducted as to whether or not the functions of proteins synthesized with a cell-free translation system using immobilized mRNA differ depending on the properties (hydrophobicity, hydrophilicity) of the surface of the beads on which the mRNA has been immobilized. The preparation of mRNA, translation and other experiment conditions were the same as in Example 1.

(1) Expression of GFP on Surface of Solid Phase by Immobilized mRNA

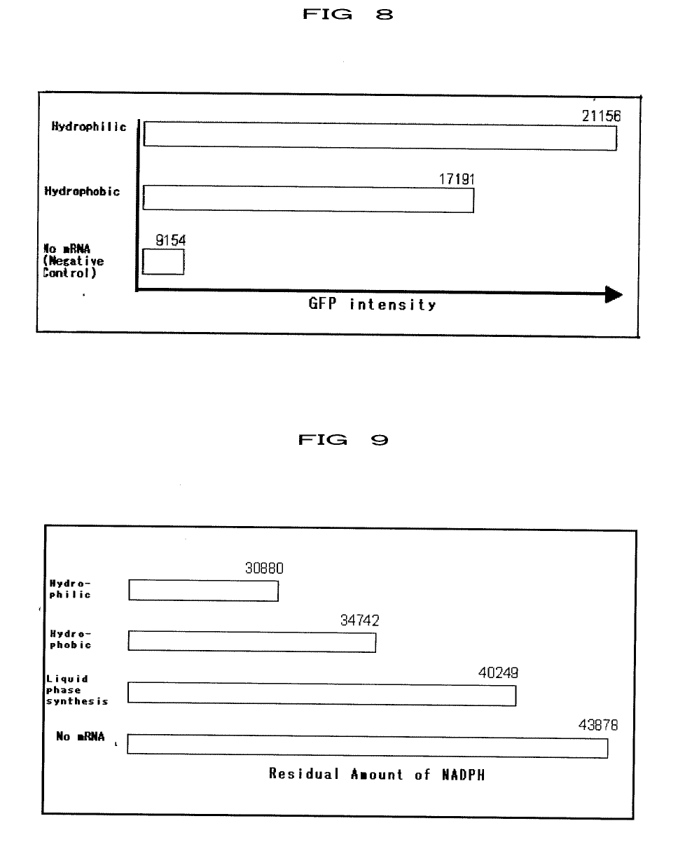

2 pmol of mRNA encoding GFP having a stop codon were linked with 3 pmol of mRNA-immobilized linker followed by immobilizing on two types of magnetic beads coated with streptoavidin (hydrophilicity: M-270, hydrophobicity: M-280, both available from Dynal) via biotin attached to the linker. When synthesizing using 40 μl of a wheat germ cell-free translation system for 10 minutes at 25° C., care was taken so that the beads did not precipitate by rotating with a rotor. Following the reaction, the reaction was stopped by allowing to stand undisturbed on ice for 5 minutes. Next, a sample and an equal amount of RNase-free water were added for the purpose of measurement, and fluorescence intensity of the GFP was measured with a microplate reader (FluPolo, Takara). Those results are shown in FIG. 8.

### (Results)

As shown in FIG. 8, the intensity of the GFP synthesized on the hydrophilic beads was about 1.5 times higher than the intensity of the GFP synthesized on the hydrophobic beads. It was thus determined that in the case of synthesizing on a solid phase, hydrophilic beads are advantageous in terms of GFP folding and so on.

### (2) In Vitro Virus Synthesis of Aldehyde Reductase (AKR) on a Solid Phase Surface

10 pmol of AKR mRNA (without a stop codon) were linked with 15 pmol of puromycin linker in accordance with Example 1 followed by immobilizing on 1 mg of two types of magnetic beads (hydrophilicity: M-270, hydrophobicity: M-280, both available from Dynal). Next, the reaction was carried out while preventing the beads from precipitating by using a rotor in 80 μl of a reaction system consisting of a wheat germ cell-free translation system for 25 minutes at 30° C. Next, MgCl₂ and KCl were added to the reaction solution to final concentrations of 90 mM and 630 mM, respectively, followed by reacting for 2 hours at 37° C. to promote formation of mRNA-protein. The AKR immobilized on the beads was separated from the beads with RNase T1, and the enzyme activity of the AKR was measured by measuring the concentration of unreacted NADPH with a microplate reader in the same manner as Example 1. (The amount of NADPH becomes lower the higher the activity of the AKR.) Those results are shown in FIG. 9.

### (Results)

As shown in FIG. 9, although the activity of AKR was higher when synthesized on beads as compared with synthesizing in a liquid phase, a comparison of activity between hydrophobic beads and hydrophilic beads revealed that the activity of AKR synthesized on hydrophobic beads was about 2.5 times higher than that synthesized in a liquid phase, while the activity of AKR synthesized on hydrophilic beads was about 3.5 times higher than that synthesized in a liquid phase.

### Example 4

### Effect of Differences in Distance Between Immobilized Location and Stop Codon of Immobilized mRNA on Activity of Synthesized Protein

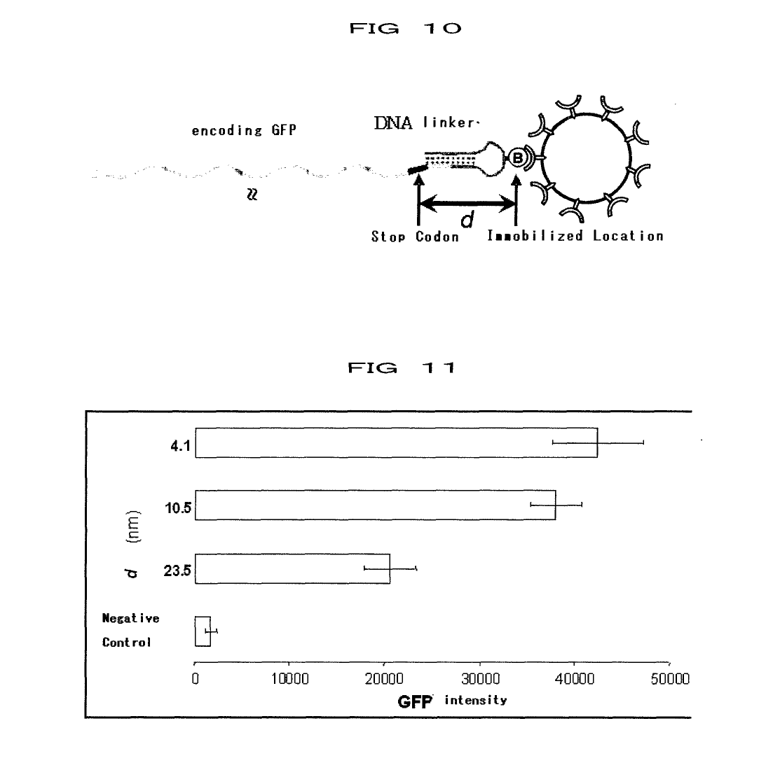

DNA linkers designed so as to have different distances (d) between the stop codon and the immobilized location of immobilized mRNA (bead surface) were prepared as shown in Table 1. The relationship between the distance (d) between the stop codon and the immobilized location of immobilized mRNA (GPF-mRNA) is shown in FIG. 10.

TABLE 1

DNA Linker Sequences and Actual Distances (d)
during Use

Distance d

| | | | |
|---|---|---|---|
| SEQ ID | Nucleo- | | |
| NO. | tides | Nm | Sequence (5′ to 3′) |

| | | | |
|---|---|---|---|
| SEQ ID | 13 | 4.1 | TAATAAGGGGGCGGCGGGGAAA |
| NO. 10 | | | |
| SEQ ID | 32 | 10.5 | GAATTCGAGCTCCGTCGACAGGAC |
| NO. 11 | | | GGGGGGCGGCGGGGAAA |
| SEQ ID | 70 | 23.5 | GAATTCGAGCTCCGTCGACAAGCTTGCG |
| NO. 12 | | | GCCGCACTCGAGCATTATTATTATTAT |
| | | | TAAGGACGGGGGGCGGCGGGGAAA |

5 pmol of mRNA encoding GFP were linked with 7.5 pmol each of the three types of DNA linkers shown in Table 1 in accordance with Example 1 followed by immobilizing on 400 μg of streptoavidin-magnetic beads (Dynal-270, Dynal). The reaction was carried out in a wheat germ cell-free translation system (40 μl) while rotating with a rotor for 90 minutes at 25° C. The reaction was stopped by allowing the reaction solution to stand undisturbed for 5 minutes on ice followed by diluting to twice the volume with sterilized water and measuring the fluorescence of the GFP with a microplate reader. Those results are shown in FIG. 11.

### (Results)

As shown in FIG. 11, it was determined that the shorter the distance (d) the immobilized location and stop codon of the immobilized mRNA, the stronger the fluorescence intensity of the GFP. More specifically, activity was determined to improve roughly two-fold in the case of the distance d being shortened to 4.1 nm as compared with a distance d of 23.5 nm.

On the basis of these findings, the presence of the stop codon of the mRNA at a location near to the surface of a hydrophilic solid phase was found to be effective for protein folding.

### INDUSTRIAL APPLICABILITY

As has been described above, according to the protein synthesis method of the present invention, a desired protein can be efficiently synthesized so that it is properly folded so as to demonstrate a function thereof. This type of protein synthesis method of the present invention is effective for large-volume synthesis of biopharmaceuticals and other useful proteins.

1. A protein synthesis method for synthesizing a desired protein so that the protein is properly folded so as to demonstrate a function thereof, the method comprising: contacting a translation system with a solid phase-immobilized mRNA in which the 3′-terminal of mRNA encoding that protein is immobilized on a solid phase including biotin.

2. The protein synthesis method according to claim 1, wherein the translation system is a cell-free translation system.

3. The protein synthesis method according to claim 1, wherein the solid phase-immobilized mRNA is immobilized on the solid phase through a linker at the 3′-terminal of the mRNA.

4. The protein synthesis method according to claim 1, wherein the solid phase-immobilized mRNA is bound to the solid phase through a solid phase binding site provided on the linker.

5. The protein synthesis method according to claim 3, wherein the linker contains, as a main backbone thereof, a polynucleotide, polyethylene, polyethylene glycol, polystyrene, peptide nucleic acid or combination thereof.

6. The protein synthesis method according to claim 1, wherein the distance between the stop codon of the solid phase-immobilized mRNA and the immobilized location of the mRNA is 10 nm or less.

7. The protein synthesis method according to claim 1, wherein the solid phase is selected from styrene beads, glass beads, agarose beads, Sepharose beads, magnetic beads, glass substrate, silicon substrate, plastic substrate, metal substrate, glass container, plastic container and membrane.

8. The protein synthesis method according to claim 1, wherein the surface of the solid phase is hydrophilic.

9. A solid phase-immobilized mRNA for synthesizing a desired protein so that the protein is properly folded so as to demonstrate a function thereof, wherein mRNA encoding the desired protein is immobilized on a solid phase through a linker.

10. The solid phase-immobilized mRNA according to claim 9, wherein the solid phase-immobilized mRNA is immobilized on the solid phase through a linker at the 3′-terminal of the mRNA.

11. The solid phase-immobilized mRNA according to claim 9, wherein the solid phase-immobilized mRNA is bound to the solid phase through a solid phase binding site provided on the linker.

12. The solid phase-immobilized mRNA according to claim 9, wherein the linker contains, as a main backbone thereof, a polynucleotide, polyethylene, polyethylene glycol, polystyrene, peptide nucleic acid or combination thereof.

13. The solid phase-immobilized mRNA according to claim 9, wherein the distance between the stop codon of the solid phase-immobilized mRNA and the immobilized location of the mRNA is 10 nm or less.

14. The solid phase-immobilized mRNA according to claim 9, wherein the solid phase is selected from styrene beads, glass beads, agarose beads, Sepharose beads, magnetic beads, glass substrate, silicon substrate, plastic substrate, metal substrate, glass container, plastic container and membrane.

15. The solid phase-immobilized mRNA according to claim 9, wherein the surface of the solid phase is hydrophilic.

16. A protein synthesis apparatus for synthesizing a desired protein so that the protein is properly folded so as to demonstrate a function thereof, the apparatus comprising: a solid phase-immobilized mRNA in which mRNA encoding the desired protein is immobilized on a solid phase through a linker.
